(19) United States
(12) Patent Application Publication
(10) Pub. No.: US 2008/0183202 A1
(43) Pub. Date: Jul. 31, 2008

(54) MINIMALLY INVASIVE RECTAL BALLOON APPARATUS WITH PRESSURE RELIEVING LUMEN

(76) Inventor: John Isham, Houston, TX (US)

Correspondence Address: EGBERT LAW OFFICES, 412 MAIN STREET, 7TH FLOOR, HOUSTON, TX, 77002, US

(21) Appl. No.: 11/966,544

(22) Filed: Dec. 28, 2007

Related U.S. Application Data
(63) Continuation-in-part of application No. 11/933,018, filed on Oct. 31, 2007.
Application No. 11/933,018 is a continuation-in-part of application No. 11/623,702, filed on Jan. 16, 2007.

(51) Int. Cl. A61M 29/02

(52) U.S. Cl. 606/192

(57) ABSTRACT

A rectal balloon apparatus has a shaft with a fluid passageway extending therethrough. A balloon is affixed over an end of the shaft such that the fluid passageway communicates with an interior of the balloon. A lumen extends along the shaft so as to allow bodily gas pressure to pass therethrough. The balloon has a laterally flat surface thereon when inflated. A sleeve is positioned over a portion of the shaft. The lumen is affixed to a surface of the sleeve. The sleeve has a first portion and a second portion frangibly connected together.

### CROSS-REFERENCE TO RELATED U.S. APPLICATIONS

The present application is a continuation-in-part of U.S. application Ser. No. 11/933,018, filed on Oct. 31, 2007, entitled “Minimally Invasive Rectal Balloon Apparatus”, presently pending. U.S. Application Ser. No. 11/933,018 is a continuation-in-part of U.S. application Ser. No. 11/623,702, filed on Jan. 16, 2007, entitled “Minimally Invasive Rectal Balloon Apparatus”, presently pending.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to an apparatus for facilitating performance of diagnostic and therapeutic treatment of disease. Particularly, the present invention relates to rectal balloons that are used for immobilizing the region surrounding the prostate during pre-treatment simulation and target localization, as well as during the delivery of radiation therapy to treat prostate cancer. More particularly, the present invention relates to minimally invasive rectal probes fitted with a lumen for relieving bodily gas pressure.

2. Description of Related Art Including Information Disclosed Under 37 CFR 1.97 and 37 CFR 1.98

Treatment of prostate cancer using radiation therapy is difficult due to the prostate's position near radiation-sensitive tissues, and is further complicated by prostate motion. Adenocarcinoma of the prostate commonly occurs in the posterior portion of the prostate gland which is in very close proximity to the rectal wall. To plan external beam radiation treatment, urethrograms, CT scans and magnetic resonance imaging (MRI) have all been used to visually localize the prostate, as well as the normal critical structures in the surrounding area.

U.S. Pat. No. 5,476,095, issued on Dec. 19, 1995 to Schnall et al., describes an insertable pickup probe for use in providing diagnostic MRI images. The pickup probe in its preferred embodiment is for use in imaging the male prostate and comprises an elongated shaft supporting an inflatable patient interface balloon at its distal end. The interface balloon comprises an inner balloon and an outer balloon, between which a receiving coil is positioned. A lumen for air supply is provided in the shaft for expanding the inner balloon against the outer balloon to place the receiving coil in close proximity to the area of interest in order to provide MRI images.

Typically, the planning of radiation therapy for the treatment of prostate cancer involves the patient undergoing a CT-based simulation scan of the pelvis to determine the location of the prostate gland. In the simulation phase, the patient is placed on CT equipment that is preferably similar to the radiation treatment equipment (except that it does not generate the high energy radiation beam). The simulation equipment is positioned to simulate the delivery of the sequence of treatment beams prescribed by the treating oncologist. Normally, during the simulation procedure, CT images are acquired. These CT images allow the oncologist to locate the position of the tumor and help to facilitate the composition of a radiation treatment plan. This treatment plan delineates the positions of the radiation equipment components for delivery of the treatment beams.

During the actual treatment phase, the patient is placed in the same position on the treatment equipment as in the simulation scans. Radiation-emitting devices are generally known and used for radiation therapy in the treatment of patients. Typically, a radiation therapy device includes a gantry, which can be swiveled around a horizontal axis of rotation in the course of a therapeutic treatment. A linear accelerator is located in the gantry for generating a high-energy radiation beam for therapy. During treatment, the radiation beam is provided by this equipment and is delivered to the patient at the precise location as delineated by the physician during simulation. A further feature of radiation therapy involves portal images, which are commonly used in radiation therapy to verify and record the patient tumor location. Portal images include manual (film) and electronic images (EPI) taken before and/or after the treatment.

During external beam radiation therapy, radiation is directed to the target prostate which is near the rectal wall. A misdirected radiation beam may perforate the rectal wall causing radiation proctitus (rectal bleeding). This toxicity is related to the total radiation dose prescribed and the volume of the anterior rectal wall receiving a high radiation dose. A major factor limiting radiation oncologists' attempts to reduce the volume of the anterior rectal wall receiving a high radiation dose is the position of the prostate gland as well as the intrinsic motion up to 5 mm in the anterior to posterior direction caused by rectal peristalsis. Accordingly, oncologists generally will add a margin to the radiation field in order to ensure that the entire prostate gland receives the prescription dose. This margin is typically on the order of 5 to 15 mm. As a consequence, lower doses of radiation may need to be used so as not to overexpose radiation sensitive structures. However, this may lead to inadequate radiation treatment and a higher probability of local cancer recurrence.

U.S. Patent Publication No. 2003/0028097, published on Feb. 6, 2003 to D'Amico et al., describes an immobolizer probe system and method. This system has an insertable probe for immobilizing a region of interest during staging and radiation therapy thereof. In particular, this device uses a balloon having a rectangular cross section connected to a shaft. The shaft extends to an end of the balloon so as to allow fluid flow through an interior of the shaft and into the balloon so as to selectively inflate the balloon once the balloon is installed into the rectal cavity. The balloon, shaft and handle are bonded together so that they move radially as a single unit when torque is applied. A syringe is provided which connects the shaft and serves as an air pump to deliver a volume-limited amount of air to the air lumen of the shaft to the balloon. A stop cock is provided to maintain the air within the balloon.

One of the problems with the subject of U.S. Patent Publication No. 2003/0028097 is the discomfort associated installing the rectal balloon within the rectal cavity. In particular, a relatively sturdy and wide diameter shaft is connected to a relatively large thick-walled balloon. Because the balloon is not supported by anything other than by the shaft, the balloon is formed of a relatively rugged and thick material. Because of the relatively large size of the shaft and the thick material of the rectangular-cross section balloon, the installation of the rectal balloon creates a large amount of discomfort for a patient. It is often difficult for the medical personnel to know exactly how far within the rectum the balloon has been installed. It is difficult to achieve a standardized and fixed position of the balloon during each and every use. The medical personnel must generally approximate the desired position of the balloon within the rectal cavity. As such, a need has developed whereby the rectal balloon can be formed of a minimal diameter shaft and of a balloon of relatively thin material.

When the rectal balloon of U.S. Patent Publication No. 2003/0028097 is in an inflated condition, the outer surface is generally round. As such, the prostate will tend to balance on the curved surface rather than properly seating thereon. Since seating is important for proper use, this device requires that the physician approximate a seated position rather than providing any feedback of the seated position. When the balloon is in a curved inflated condition, the prostate will have a tendency to slide to one side of the balloon. As such, a need developed to provide a rectal balloon that retains the prostate in a proper seated position when the balloon is in a fully inflated condition.

The Inventor is the owner of U.S. application Ser. No. 11/623,702, filed on Jan. 16, 2007. This application describes a rectal balloon apparatus which has a shaft with a fluid passageway extending therethrough, a balloon affixed over an end of the shaft such that the fluid passageway communicates with the interior of the balloon, and a sleeve slidably affixed over the shaft. This sleeve is movable between a first position overlying the balloon and a second position away from the balloon. The sleeve has an aperture at an end thereof such that the balloon extends outwardly of this aperture.

The Inventor is also the owner of U.S. application Ser. No. 11/933,018, filed on Oct. 31, 2997, which is a continuation-in-part of U.S. application Ser. No. 11/623,702. This application additionally describes the balloon as having a laterally flat surface thereon when inflated. There is a longitudinal groove extending along the laterally flat surface. Together, the laterally flat surface and the groove enhance the seating of the prostate on the balloon. Additionally, the application describes a valve assembly on the end of the shaft opposite the balloon. A fluid introducing means is removably attached to the valve assembly to introduce fluid into the valve assembly and move the balloon from the non-inflated condition to the inflated condition.

A significant cause of patient discomfort associated with rectal balloons of the prior art is the buildup of gas pressure when the balloon is inserted into the rectum. This buildup of gas pressure can also affect the ability to properly seat the balloon in a desired position. As such, a need has developed to provide a rectal balloon apparatus that allows for the release of bodily gas pressure.

It is an object of the present invention to provide a rectal balloon apparatus which is easy to use and easy to install.

It is another object of the present invention to provide a rectal balloon apparatus that has a mechanism to relieve bodily gas pressure prior to or during the inflation of the balloon.

It is another object of the present invention to provide a rectal balloon apparatus whereby the position of the balloon can be easily ascertained by medical personnel.

It is a further object of the present invention to provide a rectal balloon apparatus which maximizes the comfort to the patient.

It is a further object of the present invention to provide a rectal balloon apparatus which has better holding stability when inflated.

It is a further object of the present invention to provide a rectal balloon apparatus which has a flexible shaft.

It is another object of the present invention to provide a rectal balloon apparatus which can be inflated to several configurations for different medical purposes.

It is another object of the present invention to provide a rectal balloon apparatus which enhances the ability to properly seat the prostate on a flat surface of the rectal balloon.

It is a further object of the present invention to provide a rectal balloon apparatus which avoids potential allergic reactions.

It is still a further object of the present invention to provide a rectal balloon apparatus which is easy to manufacture and relatively inexpensive.

These and other objects and advantages of the present invention will become apparent from a reading of the attached specification and appended claims.

### BRIEF SUMMARY OF THE INVENTION

The present invention is a rectal balloon apparatus that comprises a shaft having a fluid passageway extending therethrough and a balloon affixed over an end of the shaft such that the fluid passageway communicates with an interior of the balloon. The balloon is movable from a non-inflated condition to a first inflated condition. The apparatus also comprises a lumen extending along the shaft so as to have an end opening at or beyond an end of the balloon. The balloon has a laterally flat surface when in the first inflated condition. The balloon is movable from the first inflated condition to a second inflated condition. In the second inflated condition, the balloon has both a laterally flat surface and a bulbous portion. The first inflated condition is reached when the balloon has approximately 100 cc of fluid therein. The second inflated condition is reached when the balloon has approximately 140 cc of fluid therein.

In the present invention, a groove is formed in the laterally flat surface. The groove extends longitudinally along a portion of the laterally flat surface. There is a sleeve slidably affixed over the shaft and movable between a first position overlying the balloon and a second position away from the balloon. The sleeve has a first portion frangibly joined to a second portion. The first portion has a tab extending outwardly from an end of the first portion. The second portion also has a tab extending outwardly from the second portion.

In the present invention, the shaft is made of a flexible material. There is a valve means affixed to the shaft at an end opposite the balloon. The valve means is for selectively allowing a fluid to pass through the shaft and to the balloon so as to move the balloon from a non-inflated condition to an inflated condition. The valve means has a port thereon. A fluid introducing means is removably connected to the port and can move the balloon from a non-inflated condition to an inflated condition. The non-inflated condition can be a vacuum such that the balloon resides tightly over the shaft.

In the preferred embodiment of the present invention, the lumen is affixed to an exterior of the sleeve. The lumen extends along the sleeve so as to have an end opening at or beyond an end of the balloon.

In a first alternative embodiment of the present invention, the lumen is affixed to an exterior of the shaft. The lumen extends past an end of the shaft and is in communication with the exterior of the balloon.

In a second alternative embodiment of the present invention, a one-way valve means is formed within the shaft. The one-way valve means selectively allows bodily gas to pass from the exterior of the balloon into the fluid passageway. The valve means prevents fluid or gas from passing from the fluid passageway to the exterior of the balloon.

### BRIEF DESCRIPTION OF THE SEVERAL VIEWS OF THE DRAWINGS

FIG. 1 is a side elevational view, partially transparent, which shows the rectal balloon apparatus in an uninflated condition.

FIG. 2 is an isolated view of the sleeve as used in the rectal balloon apparatus.

FIG. 3 is a side elevational view of the rectal balloon apparatus of the present invention in the first inflated condition.

FIG. 4 is a side view, partially transparent, of the balloon of the rectal balloon apparatus in the first inflated condition.

FIG. 5 is a top view of the inflated balloon as used in the rectal balloon apparatus of the present invention.

FIG. 6 is a side view, partially transparent, of the balloon of the rectal balloon apparatus in the second inflated condition.

FIG. 7 is a side view, partially transparent, of a first alternative embodiment of the balloon of the rectal balloon apparatus of the present invention.

FIG. 8 is a side view, partially transparent, of a second alternative embodiment of the balloon of the rectal balloon apparatus of the present invention.

FIG. 9 is an isolated view showing the valve assembly of the rectal balloon apparatus in an aligned configuration.

### DETAILED DESCRIPTION OF THE INVENTION

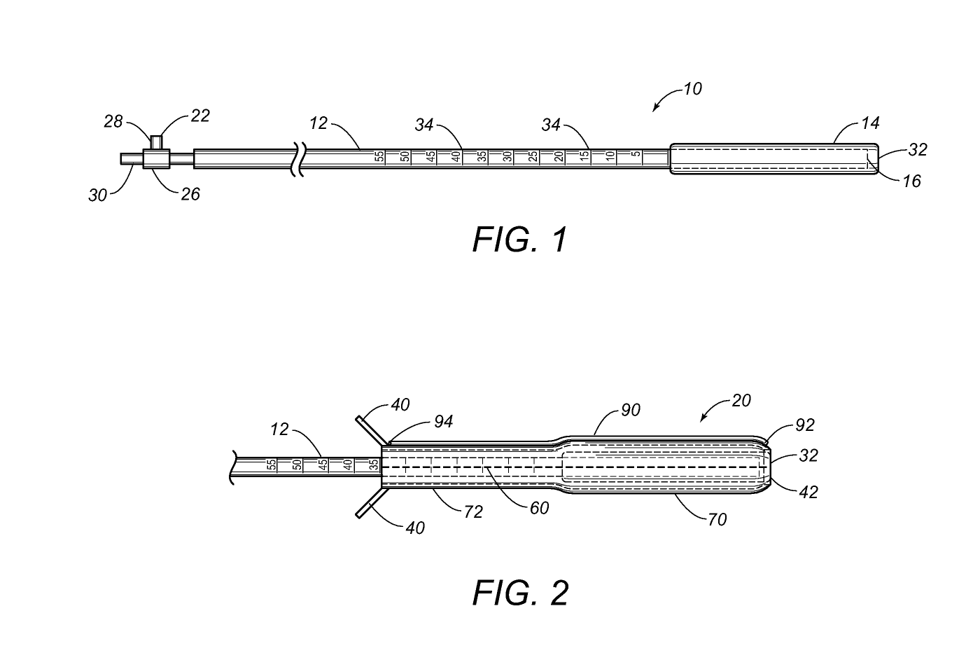

Referring to FIG. 1, there is shown the rectal balloon apparatus 10 in accordance with the preferred embodiment of the present invention. The rectal balloon apparatus 10 includes a shaft 12 having a fluid passageway extending therethrough. A balloon 14 is affixed over the end 16 of the shaft 12. The balloon 14 is shown in an uninflated condition. The fluid passageway of the shaft 12 can communicate with the interior of the balloon 14.

The shaft 12 is a generally longitudinal shaft which has the fluid passageway extending through the center thereof. The shaft 12 is made of a flexible material. A valve assembly 22 is affixed to the shaft 12 opposite the balloon 14. The valve assembly 22 can have a variety of configurations. FIG. 1 illustrates the valve assembly 22 as an inline valve assembly configuration. The valve assembly 22 may also be an angled valve assembly configuration. The valve assembly 22 includes a stop cock 26. A valve 28 facilitates the ability of the stop cock 26 to open and close so as to selectively allow the fluid to pass into the shaft 12. A port 30 allows the valve assembly 22 to be connected to a supply of the fluid. When the stop cock 26 is opened by the rotation of the valve 28, the fluid will flow through the valve assembly 22, through the interior passageway of the shaft 12, and into the interior of the balloon 14. The valve 28 can then be closed so as to maintain the inflated configuration of the balloon 14. When the procedure is finished and the fluid needs to be removed from the balloon 14, the valve 28 of stop cock 26 can then be opened so as to allow for the release of fluid therethrough.

The opposite end 16 of the shaft 12 contacts the end 32 of the balloon 14. The end 16 is suitably curved or dome-shaped so as to allow the shaft 12 to facilitate the introduction of the balloon 14 into the rectal cavity. The shaft 12 has indicia 34 formed therealong. It can be seen that the indicia 34 has numerical references associated therewith. These numerical references are indicative of the distance that the balloon 14 has been inserted into the rectum. As such, the indicia 34 provide a clear indication to the medical personnel of the desired location of the rectal balloon 14.

FIG. 2 is an isolated view of the sleeve 20 in the preferred embodiment of the present invention. The sleeve 20 has a first portion 70, a second portion 72, and a lumen 90. The lumen 90 is affixed to the first portion 70 of the sleeve 20. The lumen 90 is a hollow passageway. When the balloon 14 is inserted into the rectum, gas pressure can build up behind the balloon 14. The gas can pass into the lumen 90 through first opening 92 and exit the body through second opening 94, thereby relieving the discomfort caused by the gas buildup. For clarity, the sleeve 20 is not shown covering the balloon 14 in FIG. 1.

Importantly, in FIG. 2, it can be seen that the sleeve 20 has a frangible surface 60 extending longitudinally therealong joining first portion 70 and second portion 72. The frangible surface 60 is arranged such that when the sleeve 20 is pulled toward the handle 22, proper pulling forces can cause the sleeve 20 to split longitudinally therealong so that the sleeve 20 can be permanently removed from the shaft 12. The sleeve 20 has tabs 40 attached to first portion 70 and second portion 72 at an end opposite the balloon 14 to be used when pulling the sleeve 20. The sleeve 20 also has an aperture 42 formed at an end opposite to the tabs 40. The aperture 42 allows the end 32 of the balloon 14 to extend slightly outwardly therefrom. As such, the end 42 will create a smooth contour, in combination with the end 32, so as to facilitate the ease of insertion of the apparatus 10.

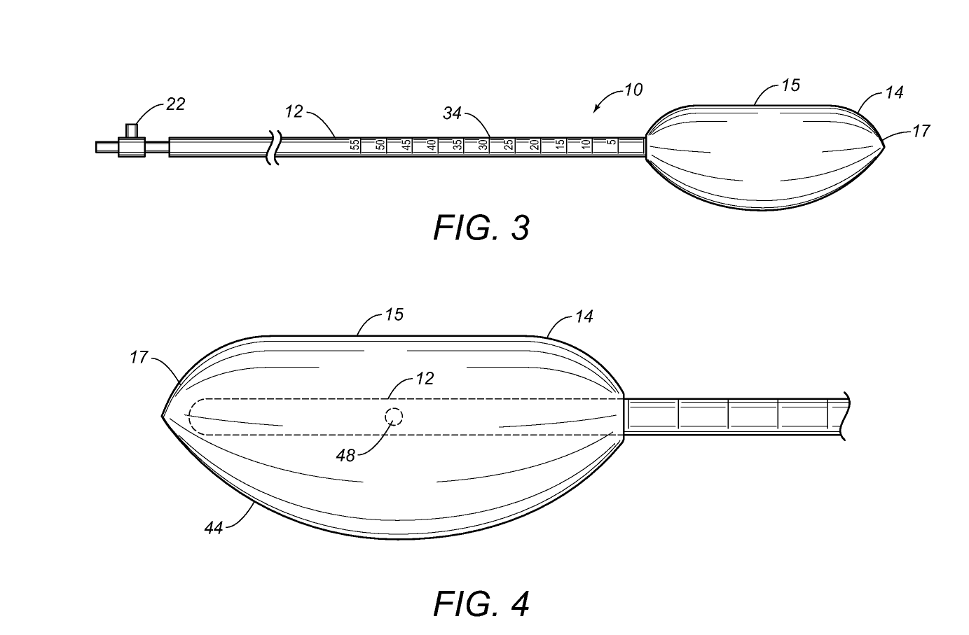

FIG. 3 illustrates the apparatus 10 after the apparatus has been installed within the rectum. The fluid can be introduced through the valve assembly 22 and through the interior passageway of the shaft 12 so as to inflate the balloon 14. The balloon 14 has a seating area 15 so that the prostate can be properly positioned thereon. The balloon 14 has a head portion 17 adjacent the tip of the balloon 14 opposite the shaft 12. When the balloon 14 is installed and inflated, the prostate will reside on the flat surface 15 in a seated position. The head portion 17 will abut the tip of the prostate. After the procedure has been completed, the balloon 14 can be deflated and easily pulled outwardly of the rectum in its deflated condition.

FIG. 4 is an isolated view of the balloon 14 as inflated to a first inflated condition. In this first condition, the balloon 14 has a central seating area 15, a head portion 17 and a bottom portion 44. The central seating area 15 has a lateral flatness when inflated for the prostate to rest upon. The first inflated condition is formed when approximately 100 cc of fluid are introduced into the balloon 14. The lateral flatness of the seating area 15 will prevent the prostate from sliding to one side or the other. The bottom portion 44 is rounded and contacts the rectal wall. The head portion 17 is generally V-shaped so as to facilitate easier insertion of the balloon 14. The material of the balloon 14 is formed of a non-latex material so as to avoid allergic reactions. The shaft 12 is shown extending into the interior of the balloon 12. A hole 48 is formed in the shaft 12 through which the balloon 14 is filled with fluid. The hole 48 is formed generally centrally within the balloon 14.

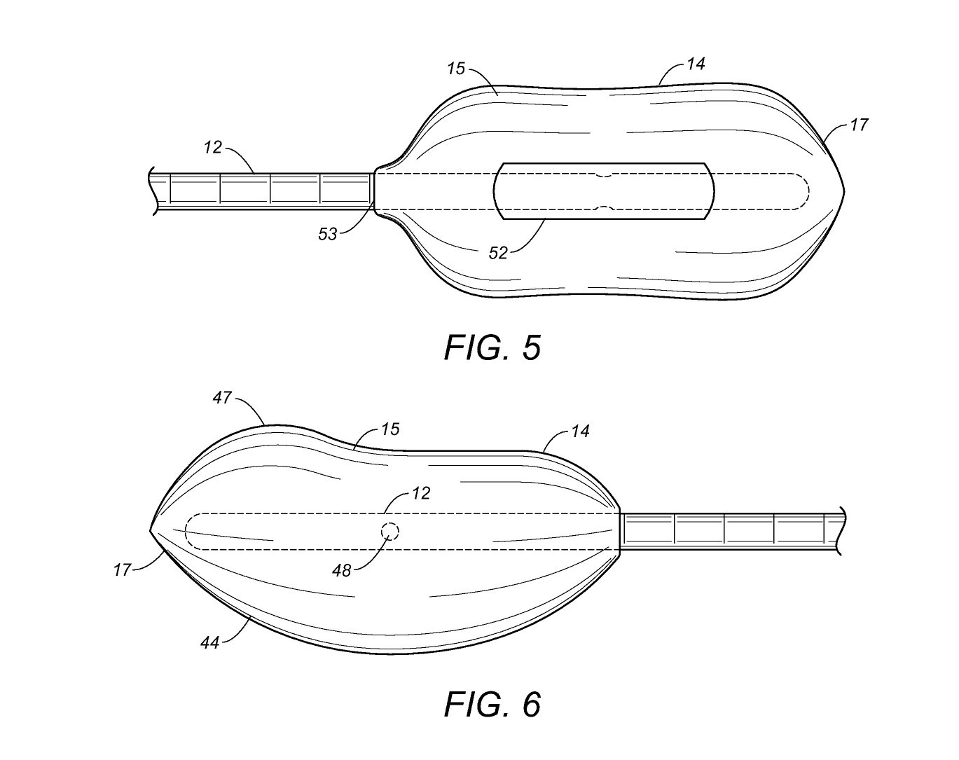

FIG. 5 is a top view of the balloon 14 from the side of the balloon 14 which engages with the prostate. Central seating area 15 is shown as having a groove 52 formed thereon. The groove 52 is generally rectangularly-shaped and engages with the tip of the prostate and reduces lateral motion. The central seating area 15 and the groove 52 enhance the holding stability of the balloon 14 of the present invention. In FIG. 5, it can also be seen that head portion 17 of the balloon 14 is generally V-shaped. This shape makes insertion of the balloon 14 into the rectum easier for medical personnel and more comfortable for the patient. The balloon 14 has a thermally welded bond 53 connecting it to the shaft 12.

FIG. 6 is an isolated view of the balloon 14 as inflated to a second inflated condition. In the second inflated condition, the balloon 14 has a bulbous portion 47 formed adjacent to the head portion 17. The balloon also has a laterally flat seating area 15. The bulbous portion 47 can be utilized in certain conditions to better isolate the prostate. Generally, the bulbous portion 47 will be formed when at least 140 cc of fluid are introduced into the balloon 14.

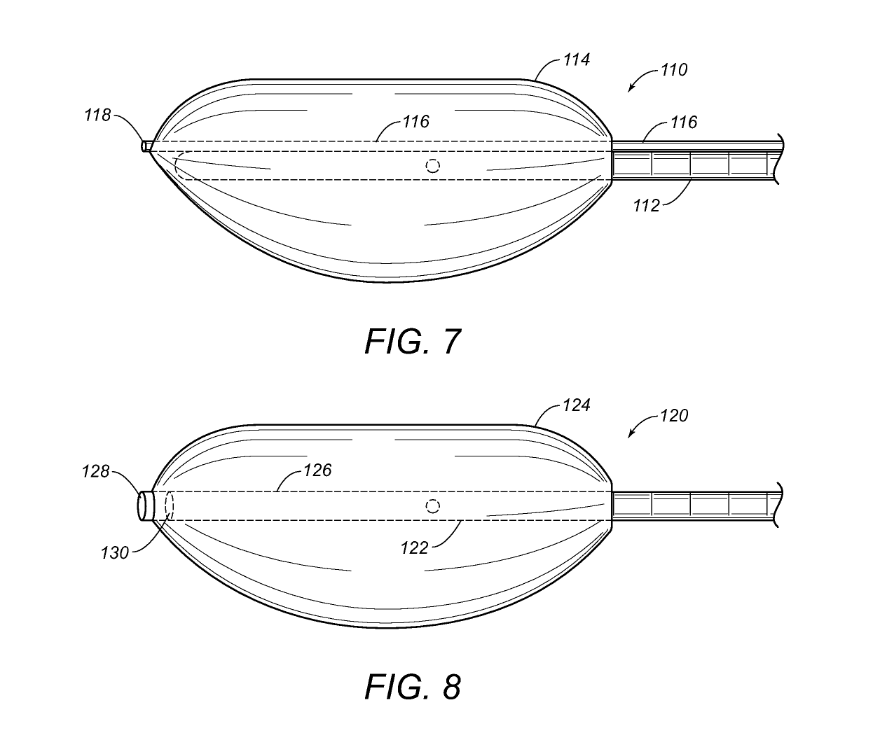

FIG. 7 is a side view of a first alternative embodiment of the balloon 114 of the rectal balloon apparatus 110 of the present invention. In addition to the features shown in FIG. 4, the balloon 114 in the first alternative embodiment has a lumen 116 affixed to a surface of the shaft 112. The lumen 116 extends to an exterior of the balloon 114. First opening 118 allows bodily gas to pass from an exterior of the balloon 114 into the lumen 116. The gas passes through the lumen 116 to another opening at the opposite end of the lumen 116, and into the atmosphere. In the first alternative embodiment, the lumen 116 would not be located on the sleeve 20, as shown in FIG. 2. In the first alternative embodiment, the sleeve 20 has all features shown in FIG. 2, absent the lumen 90 affixed to its exterior. The lumen 116 will have a portion extending interior of the balloon 114. The ends of the balloon 114 will be sealed around the lumen 116 and the shaft 112.

FIG. 8 is a side view of a second alternative embodiment of the balloon 124 of the rectal balloon apparatus 120 of the present invention. In the second alternative embodiment, the gas pressure relieving activity of the lumen 126 is integrated with the shaft 122. The shaft 122 extends to an exterior of the balloon 124, and has an opening 128 outside of the balloon 124. A one-way valve means 130 is formed within the shaft 122. The one-way valve means 130 allows bodily gas to pass from the exterior of the balloon 124, into the opening 128, and through the one-way valve means 130 into the shaft 122. The one-way valve means 130 prevents fluid or gas from escaping through opening 128 from the interior of the shaft 122. In the second alternative embodiment, the lumen 126 would not be located on the sleeve 20, as shown in FIG. 2. In the second alternative embodiment, the sleeve 20 has all features shown in FIG. 2, absent the lumen 90 affixed to its exterior. The one-way valve means 130 operates to allow bodily gas to escape through the interior of the shaft 122 when the balloon 124 is installed in the rectum. After the gas has escaped, fluid is introduced into the balloon 124 so as to inflate the balloon 124, while at the same time, closing the one-way valve means 130.

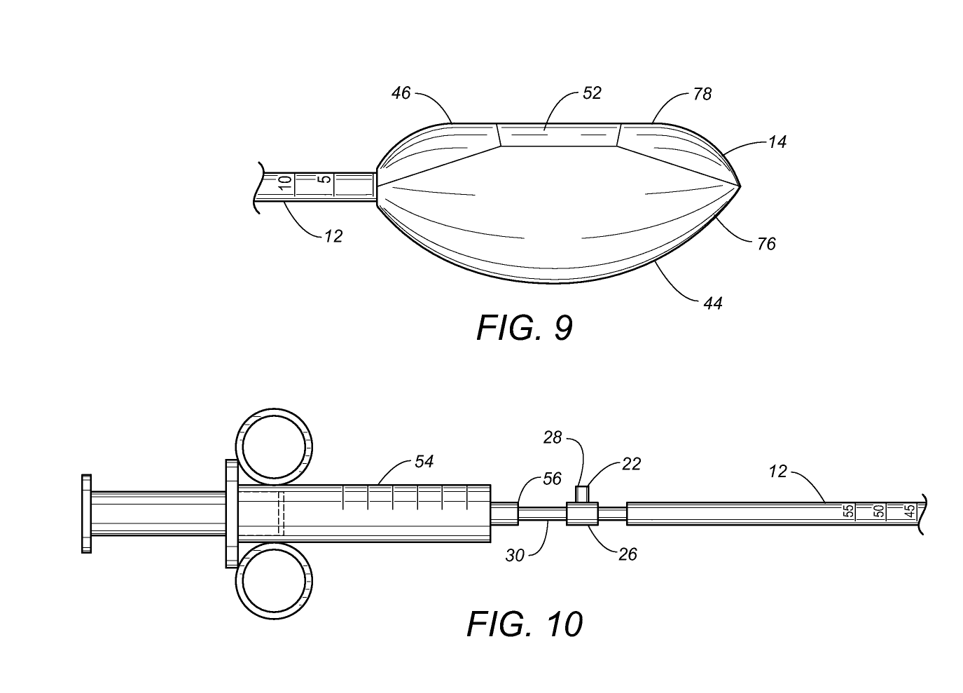

FIG. 9 shows a cross-sectional side view of the balloon 14 showing the plurality of layers which form the balloon 14. A bottom layer 76 forms the bottom portion 44 of the balloon 14. A top layer 78 forms the upper portion, including central seating area 15 and the groove 52, of the balloon 14. A middle layer 80 extends between the bottom layer 76 and the top layer 78. The middle layer 80 is connected to the top layer 78 at the groove 52.

FIG. 10 shows an isolated view of the shaft 12 of the rectal balloon apparatus 10 at an end opposite the balloon 14. The valve assembly 22 is shown in an online configuration. The valve assembly 22 is connected at one end to the shaft 12 and at the opposite end to a fluid introducing device 54. In this case, the fluid introducing device 54 is a syringe. The syringe 54 serves as a removable handle for the rectal balloon apparatus 10. An end portion 56 of the syringe 54 is removably connected to the port 30 of the valve assembly 22. When the valve 28 and the stop cock 26 are in an open position, fluid can travel from the syringe 54 into the shaft 12 and to the balloon 14. With the balloon 14 inflated, the valve assembly may be closed and the syringe 54 removed. The syringe 54 can be reconnected to the valve assembly 22 prior to removal of the balloon 14 from the rectum to aid in removing the fluid from the balloon 14.

The foregoing disclosure and description of the invention is illustrative and explanatory thereof. Various changes in the details of the illustrated construction can be made within the scope of the present claims without departing from the true spirit of the invention. The present invention should only be limited by the following claims and their legal equivalents.

I claim:

1. A rectal balloon apparatus comprising:
a shaft having a fluid passageway extending therethrough;
a balloon affixed over an end of said shaft such that said fluid passageway communicates with an interior of said balloon, said balloon movable between an inflated condition and a non-inflated condition; and
a lumen extending along said shaft so as to have an end opening at or beyond an end of said balloon.

2. The apparatus of claim 1, said balloon having a generally laterally flat surface thereon when in said inflated condition.

3. The apparatus of claim 1, said balloon movable between said first inflated condition and a second inflated condition, said balloon having both a laterally flat surface and a bulbous surface thereon when in said second inflated condition.

4. The apparatus of claim 2, said balloon having a groove formed in said laterally flat surface, said groove extending centrally longitudinally along a portion of said laterally flat surface.

5. The apparatus of claim 1, further comprising:
a sleeve slidably affixed over said shaft and movable between a first position overlying said balloon and a second position away from said balloon.

6. The apparatus of claim 5, said lumen affixed to a surface of said sleeve.

7. The apparatus of claim 5, said sleeve having a first portion frangibly joined to a second portion, said first portion having a tab extending outwardly from one end thereof, said second portion having a tab extending outwardly from one end thereof.

8. The apparatus of claim 1, said lumen affixed to said shaft and extending along an exterior of said shaft and having a portion positioned interior of said balloon, said balloon being sealed around said lumen and said shaft.

9. The apparatus of claim 1, said lumen being said fluid passageway of said shaft such that an end of said fluid passageway opens beyond said end of said balloon.

10. The apparatus of claim 9, said lumen comprising:
a one-way valving means positioned in said fluid passageway, said one-way valving means for allowing bodily gas to pass through said fluid passageway of said shaft.

11. The apparatus of claim 10, further comprising:
a fluid introducing means in fluid communication with said fluid passageway of said shaft, said one-way valving means for preventing fluid from said fluid introducing means from passing outwardly of an opposite end of said shaft, said fluid introducing means for moving said balloon from said non-inflated condition to said inflated condition.

12. The apparatus of claim 11, said shaft having a hole formed through a wall thereof so as to open to an interior of said balloon, said one-way valving means positioned between said hole and said opposite end of said shaft.

13. A rectal balloon apparatus comprising:
a shaft having a fluid passageway extending therethrough;
a balloon affixed over an end of said shaft such that said fluid passageway communicates with an interior of said balloon, said balloon movable between an inflated condition and a non-inflated condition;
a sleeve slidably affixed over said shaft and movable between a first position overlying said balloon and a second position away from said balloon; and
a lumen affixed to an exterior of said sleeve, said lumen extending along said sleeve so as to have an end opening at or beyond an end of said balloon.

14. The apparatus of claim 13, said sleeve having a first portion frangibly joined to a second portion, said first portion having a tab extending outwardly from one end thereof, said second portion having a tab extending outwardly from one end thereof, said lumen affixed to one of said first and second portions.

15. A rectal balloon apparatus comprising:
a shaft having a fluid passageway extending therethrough;
a balloon affixed over an end of said shaft such that said fluid passageway communicates with an interior of said balloon, said balloon movable between an inflated condition and a non-inflated condition; and
a lumen affixed to an exterior of said shaft and extending along said shaft, said lumen extending beyond said end of said shaft and outwardly beyond an end of said balloon, said balloon being in sealed relation over said shaft and said lumen.

16. The apparatus of claim 15, further comprising:
a sleeve slidably affixed over a portion of said shaft and over a portion of said lumen, said sleeve movable between a first position overlying said balloon and a second position away from said balloon.

17. The apparatus of claim 16, said sleeve having a first portion frangibly joined to a second portion, said first portion having a tab extending outwardly from one end thereof, said second portion having a tab extending outwardly from one end thereof.

18. A rectal balloon apparatus comprising:
a shaft having a fluid passageway extending therethrough;
a balloon affixed over a portion of said shaft such that said fluid passageway communicates with an interior of said balloon, said shaft extending outwardly beyond an end of said balloon, said balloon movable between an inflated condition and a non-inflated condition; and
a one-way valving means formed in said fluid passageway of said shaft, said one-way valving means for allowing a bodily gas to pass through said fluid passageway.

19. The apparatus of claim 18, further comprising:
a fluid introducing means in fluid communication with said fluid passageway of said shaft, said one-way valving means for preventing fluid from said fluid introducing means from passing outwardly of an opposite end of said shaft, said fluid introducing means for moving said balloon from said non-inflated condition to said inflated condition.

20. The apparatus of claim 19, said shaft having a hole formed through a wall thereof so as to open to an interior of said balloon, said one-way valving means positioned between said hole and said opposite end of said shaft.
